(19) United States
(12) Patent Application Publication
(10) Pub. No.: US 2005/0203511 A1
(43) Pub. Date: Sep. 15, 2005

(54) Orthopaedics device and system

(76) Inventors: James Wilson-MacDonald, Culham (GB); David Wycliffe Murray, Cuddesdon (GB); Thomas Allard Xander Bonnema, Hengelo (NL); Martijn Heikens, Hengelo (NL)

Correspondence Address: PAUL J. SUTTON, ESQ., BARRY G. MAGIDOFF, ESQ.;GREENBERG TRAURIG, LLP, 200 PARK AVENUE, NEW YORK, NY, 10166, US

(21) Appl. No.: 10/790,033

(22) Filed: Mar. 2, 2004

(51) Int. Cl. A61B 17/56

(52) U.S. Cl. 606/61

(57) ABSTRACT

An implantable, temporospatially dynamic, rachiorthotic orthopaedics device comprising: a unidirectional force generating means for generating a unidirectional force which acts over a range of deflection of said unidirectional force generating means; a first attachment means for attaching said unidirectional force generating means to a first vertebra; and a second attachment means for attaching said unidirectional force generating means to a second vertebra; wherein said unidirectional force is applied by said unidirectional force generating means via said first and second attachment means to said first and second vertebrae such that said first vertebra and said second vertebra are urged, over a period of time (which period of time extends beyond the end of a medical procedure to implant said orthopaedics device) and over a range of rotational, axial and/or flexional/extensional motion, towards a predetermined desired spatial relationship with respect to one another, whereby, over said period of time, said unidirectional force urges a proprioceptively neutral position of said first and second vertebrae towards a desired neutral position, and whereby a biological correction of a spinal deformity, spinal injury or other spinal disorder may be mechanically facilitated.

### FIELD OF THE INVENTION

The present invention relates to an implantable, temporospatially dynamic, rachiorthotic orthopaedics device and to an implantable, rachiorthotic, hybrid static/dynamic orthopaedics system.

### BACKGROUND ART

Abnormal spine curvatures can result from disease, weakness or paralysis of the trunk muscles, poor posture or congenital defects in vertebral anatomy. The most common deformity is an abnormal lateral and rotational deformity called scoliosis. Scoliosis is probably the longest known-of orthopaedic condition. The growing deformation of the body has amazed people throughout the ages and this has led to intensive attempts to both explain and treat the condition. Despite this, there remain many problems, caused by scoliosis, which can still not be satisfactorily solved and the causes of the most common form of scoliosis (idiopathic scoliosis) are yet to be fully discovered, Other problems involving exaggerated curvature of the spine include kyphosis (exaggerated thoracic curvature or ‘hunchback’) and lordosis (exaggerated lumbar curvature or ‘swayback’).

Low Back Pain can Occur as a Complication of Scoliosis.

Prior art treatments for abnormal spine curvatures generally follow a progression which starts with conservative approaches, involving patient physiotherapy and bracing with various forms of braces (called ‘orthoses’ in the art; from the Greek word ‘orthosis’-‘making straight’) such as the Boston Brace (see WO 97/25009) or the TriaC marketed by Somas.

If stabilisation of the condition is not achieved using conservative methods then the most widely available next step in the progression of treatment is a surgical intervention involving the implantation of an orthopaedics device which is implanted with the aim of causing spondylosyndesis (spinal fusion) of a section of the spine.

A spine fusion uses special stainless steel or titanium screws, rods, hooks, and a bone graft. The rods are attached to the spine with hooks and screws and the curved portion of the spine is forcibly straightened. Then, small strips of bone graft are placed over the spine to fuse it in a straightened position.

As the bone graft fuses over the next several months, the deformity is less likely to recur. This is a radical treatment which has clear negative implications for the future mobility of the spine.

Treatment involving spinal fusion is sometimes referred to as ‘static treatment’ in the art. Until very recent times, static treatments were the only option. Indeed much of the orthopaedic literature still refers to surgical intervention as being synonymous with spinal fusion.

The orthopaedics devices implanted for static treatment are generally in the form of vertebral staples or pedicle screws, which are attached to stiff rods. In implanting such orthopaedics devices, surgeons have to exert surprisingly large forces to the spinal column of a patient in order to bring the spine to the rod and fix it in place. The surgeon also needs to decorticate the vertebra (which means removing the hard outer surface of the bone revealing the spongy inner bone which has a better blood supply and will better encourage healing of the bone graft).

Many prior art static spinal orthopaedics devices are implanted posteriorally—using pedicle screws in the pedicles of a patients vertebra, although, more recently anterior fixation has also become more common. An example of a posterior system is given by U.S. Pat. No. 4,653,481. An example of an anterior system is given by U.S. Pat. No. 5,603,714.

However, more recently, a further type of surgical intervention has been developed—that of dynamic treatment—involving the application of an elastic force to the spine in order to hold the spine in a desired position, whilst allowing some flexibility away from the desired position.

The first dynamic product in use was the Graf ligament—described and claimed in US Re. 36,221. This is a flexible ligament—generally made of ‘Dacron’ (trade mark)—which is attached to pedicle screws in two vertebra by looping the flexible ligament around the screws.

Ligaments are very important restraining members in the musculoskeletal system. According to the Oxford Dictionary, a ‘ligament’ is a short band of tough flexible fibrous connective tissue linking bones together. Skeletal joints are kinematically constrained and stabilised by ligaments to minimise transverse or twisting displacements while maintaining rotational movements. They are subject to shock-loads during sports and exercise programs and are essentially tensile structural members, offering very little resistance in compression. They exhibit strain-hardening behaviour with a low initial modulus.

Thus the concept behind the Graf ligament is to put the spine into the desired alignment and then hold it there, in a flexible manner, using artificial ligaments.

A similar, though different, system which uses the same treatment paradigm is the Dynesys system (see EP 0 669 109). This system holds the spine, in flexible manner, in a desired alignment. This device in addition has a distracting tube around the ligament which gives further stability to the spine.

Another device very similar to the Dynesys system for stabilising the spine—holding it in a desired position—is described in U.S. Pat. No. 5,672,175.

WO 02/102259 and WO 01/45576 describe other devices for holding the spine in a desired position while allowing a small amount of constrained flexible motion.

These known ‘dynamic’ devices have had some success in treating spine curvature problems. However these devices do not provide a healing effect, but rather provide a stabilising effect. These devices stabilise the spine in a desired position, which position is set at the time of implantation of the device.

Examples of the very few orthopaedic devices which allow correction of deformity over time include orthoses (see above) and external fixators. With external fixators, screws or wires are placed into the bones and an external frame is applied to the spine. By adjusting the forces applied with time, deformity can be safely and effectively corrected. An example of this is the Ilizarov external fixator.

It is an object of the present invention to provide an orthopaedics device and system which facilitates the biological correction of spinal curvatures over time.

It is a further object of the present invention to provide an orthopaedic device and system which allows an orthopaedist to apply orthotic forces to vertebra, using an implanted device rather than an external brace.

It is a further object of the present invention to provide an orthopaedic device and system which, over time, allows the spine to heal towards a desired alignment without fusing the spine.

### SUMMARY OF THE INVENTION

The present invention seeks to overcome the problems mentioned above through provision of an implantable, temporospatially dynamic, rachiorthotic orthopaedics device according to claim 1.

Further desirable features and desirable embodiments as well as an orthopaedics system, a hybrid static/dynamic orthopaedics system and an implantation kit are detailed in claims 2 to 24.

Embodiments of the present invention provide many advantages over prior art spinal curvature correction devices.

An embodiment of the present invention allows the implantation of a device which is able to apply a force over time. This is a particular advantage in the growing child because the natural remodelling, which occurs during growth can be harnessed to help to correct the deformity with time.

An embodiment of the present invention allows complex forces to be applied between two adjacent vertebrae with oblique forces applied in some areas and axial forces in other areas of the spine, or even a combination of the two. This allows a more rational type of correction of the deformity, applying loads in the direction which they are required.

An embodiment of the present invention allows retention of the inter-vertebral discs and is thus less destructive than the present generation of anterior fusion devices. Because of this the device is easier to apply and time is saved surgically.

Most implantable devices require fusion of the spine. In scoliosis surgery this effectively means stiffened long sections of the spine. An embodiment of the present invention does not necessarily require any fusion of the spine. Thus this non-fusion device does not require bone fusion and a bone graft. This reduces morbidity in the operation, saves money and reduces hospital stay.

Part of a spinal deformity is the development of a rib bump. An embodiment of the present invention can cause a reduction of the rib hump over time because of the remodeling which it causes. This results in a more effective correction of the chest deformity than prior art devices, and reduces the likelihood of having to perform a costoplasty (operation to reduce the rib hump). This also reduces morbidity in the operation, saves money and reduces hospital stay.

Particular embodiments of the present invention are implantable using minimally invasive techniques, which further reduces morbidity in the operation, saves money and reduces hospital stay.

Particular embodiments of the present invention provide the additional advantage of being usable as a hybrid device. No such hybrid static/dynamic device exists in the art. A hybrid device allows a partial correction of the deformity during surgery with a short fusion, and implantation of a non-fusion device in the adjacent area(s) of the spine which allows correction of the remainder of the deformity over time. This results in a shorter fusion than is necessary with prior art devices.

Particular embodiments of the present invention are made of memory metal which allows forces to be determined even more accurately and for these forces to be applied as required over time.

Other aspects and advantages of the invention will be clear from a study of the following detailed description and drawings in which a particular embodiment of the invention, comprising an orthopaedics device as part of a hybrid static/dynamic orthopaedics system, is described by way of example and with reference to the accompanying drawings.

### BRIEF DESCRIPTION OF DRAWINGS

FIG. 1: A schematic representation of an embodiment of an orthopaedics device according to the invention as part of an embodiment of a hybrid static/dynamic orthopaedics system according to the invention.

FIG. 2: A different view of the embodiments of FIG. 1.

FIG. 3: A different view of the embodiments of FIG. 1.

### DETAILED DESCRIPTION

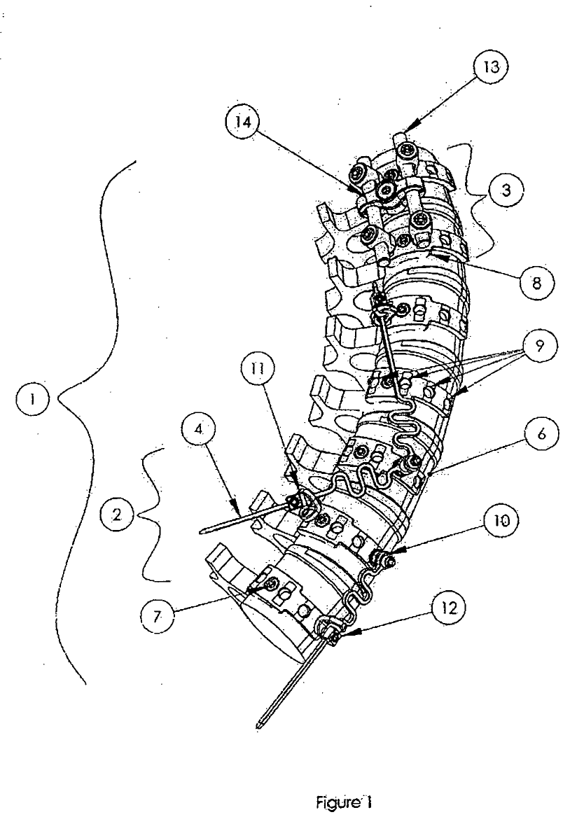

FIG. 1 shows a hybrid device, 1, with a plurality of flexible implants, 2, below and a fusion device, 3, above. Each flexible implant, 2 (also referred to as a ‘non-fusion device’ or ‘spring device’) comprises a spring, 4, made of memory metal with the typical properties of a memory metal. Plates, 5, with multiple attachments, 9, are applied to each vertebra, 6, of the curve (occasionally it might not be necessary to attach a plate to every vertebra). A plate, 5, is attached to a vertebra, 6, with screws, 7, placed through the plate, 5. The plate, 5 has small projections, 8, on the surface adjacent to the bone which stop the plate slipping. There are multiple attachment points, 9, on the plate, 5, which allow the spring, 4, to apply its force at variable angles. This allows either longitudinal compression across the motion segment or it allows oblique forces to be applied across a motion segment. The spring, 4, is attached to the plate, 5, by a universal joint, 10, at one end, which is attached firmly to the plate, 5, at that end. At the other end is a ring, 11, attached to the plate, 5, through which passes one end of the spring, 4. The spring, 4, is in turn attached to a device, 12, which allows distraction of the spring, 4, but which does not allow the spring, 4, to then slip through the ring, 11. This allows compression across the motion segment, or across more than one motion segment either longitudinally or obliquely. This allows correction of the deformity at a segmental level. The fusion device. 3, is shown in pure compression on the convexity of the curve. However the fusion device, 3, can also be used obliquely to allow rotational forces to be applied to the motion segment.

FIG. 1 shows a fusion device, 3, and non-fusion device, 2, separately, but they can be used in combination across the curve in any configuration allowed by the implant, as the surgeon wishes in order to correct a deformity.

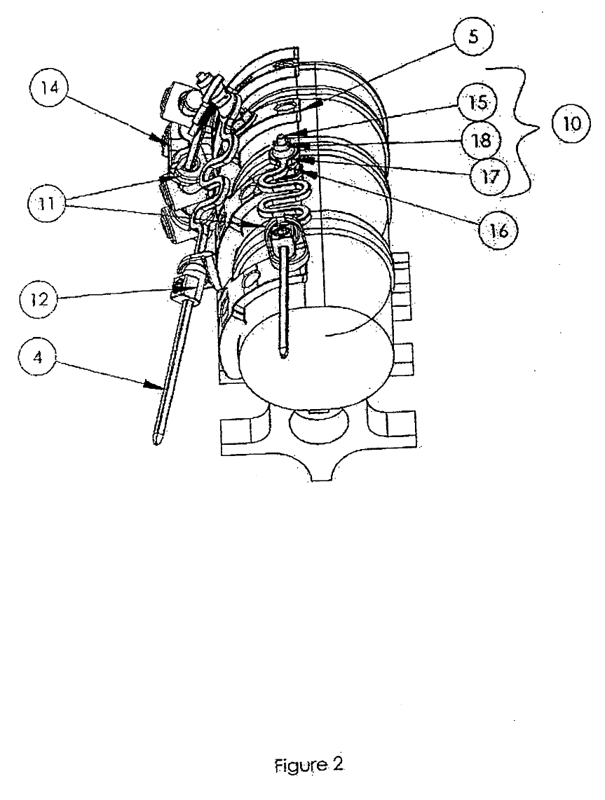

FIG. 2 shows the same implants as are shown in FIG. I but in a different projection. The bone screw, 7, is shown in some detail attaching the plate, 5, to the vertebra, 6. There may be one, two or more screws, 7, placed at each plate. The non-fusion device, 2, is shown in pure compression and as an oblique implant.

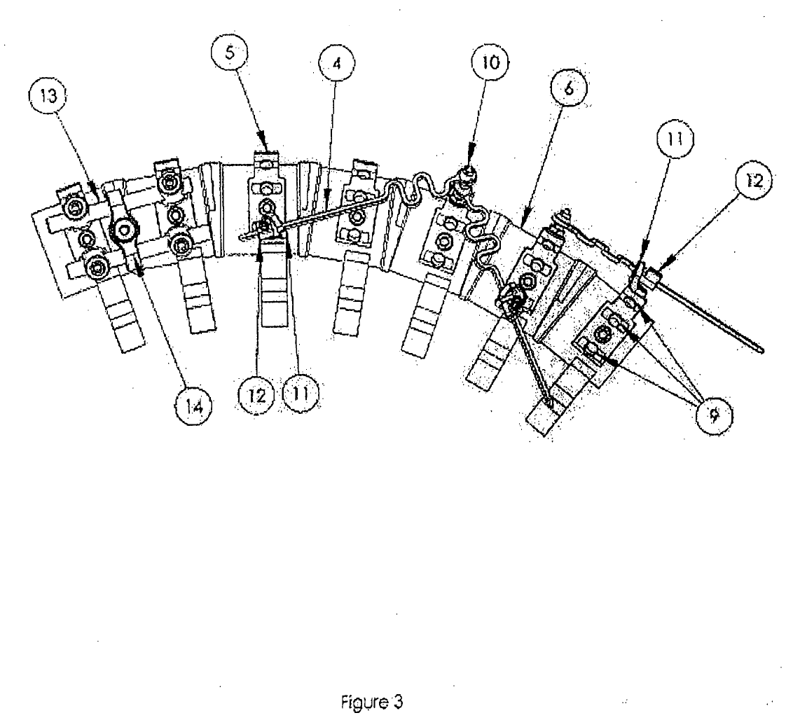

FIG. 3 shows the same implants as are shown in FIGS. 1 and 2 in a different projection. The non-fusion device, 2, has been placed across one motion segment (an intermediate device), across two motion segments (the left hand non-fusion device), and in anteriorly applying a kyphotic force (compression across the front of the spine). The fusion device, 3, is again shown in compression laterally with two rods, 13 and a cross link, 14. The fission device, 3, may also be used obliquely (not shown) to allow rotational forces to be applied across a single motion segment to allow correction of rotational deformity. The fusion device, 3, can also be used with a single rod, 13, or as a pair of rods, 13, applied obliquely.

All of the figures show a device which consists of a base plate, 5, with projections, 8, which is attached to the adjacent vertebra, 6, with either one or two screws, 7. Plates, 5 are applied across the deformed part of the spine but may not be used at every level. The screws, 7, are threaded and may allow some bone in-growth.

The springs, 4, or rods, 13, are attached to the plate. This is done by way of a pin, 15, which with its small base plate, 16, can be attached to the plate, 5. This then allows the spring, 4, to be attached to the plate, 5. There is a ring, 17, at one end of the spring, 4. This is attached to a spherically-formed member, 18, which in turn is attached to the pin, 15 to form a type of universal joint. The spherically formed member, 18, slides over or screws onto the pin, 15, and is firmly attached to the pin, 15. The other end of the spring, 4, is passed through a ring, 11, which in turn is attached to the adjacent plate, 5. A clamping device, 12, is used which clamps on to the spring, 4, and only allows the passage of the spring, 4, in one direction through the clamp. This then allows distraction of the spring, 4, and compression between the two ends of the spring, 4.

The spring, 4, is made of memory metal and the features of the spring, 4, are used to produce an optimal force across the motions segment(s).

The figures also show rods, 13, applied in pure compression across the motion segment. The rods, 13, could also be applied obliquely across a motion segment.

The rods, 13, and springs, 4, could be used in any combination chosen by a surgeon to maximise correction of a curve and prevention of progression of a deformity.

The implants can be applied across on segment or multiple segments.

This device can also be used in the management of low back pain. It allows a stabilisation of motion segment(s), in order to reduce low back pain.

Another use would be in spinal fusion surgery to “top off” a long fusion. The device can be used as a posterior non-fusion device to treat spinal deformity (either scoliosis or kyphosis).

The device can also be used to assist posterior spinal fusion.

Many further modifications and variations are possible within the context of the invention. The above described embodiment is described for illustrative purposes only and is not intended to limit the scope of the invention, that being determined by the appended claims.

1. An implantable, temporospatially dynamic, rachiorthotic orthopaedics device comprising:
a unidirectional force generating means for generating a unidirectional force which acts over a range of deflection of said unidirectional force generating means;
a first attachment means for attaching said unidirectional force generating means to a first vertebra; and
a second attachment means for attaching said unidirectional force generating means to a second vertebra;
wherein said unidirectional force is applied by said unidirectional force generating means via said first and second attachment means to said first and second vertebrae such that said first vertebra and said second vertebra are urged, over a period of time (which period of time extends beyond the end of a medical procedure to implant said orthopaedics device) and over a range of rotational, axial and/or flexional/extensional motion, towards a predetermined desired spatial relationship with respect to one another,
whereby, over said period of time, said unidirectional force urges a proprioceptively neutral position of said first and second vertebrae towards a desired neutral position, and whereby a biological correction of a spinal deformity, spinal injury or other spinal disorder may be mechanically facilitated.

2. An orthopaedics device according to claim 1 in which said unidirectional force is insufficient to cause said first and second vertebrae to attain said predetermined desired spatial relationship at the time of implantation.

3. An orthopaedics device according to claim 1 in which the magnitude of said unidirectional force is in the range of 0N to 200N

4. An orthopaedics device according to claim 1 in which at least one of said first and second attachment means comprises a mobile joint chosen from the group consisting of a ball-and-socket joint or a hinge joint or a saddle joint or a pivot joint or a gliding joint or a condyloid joint.

5. An orthopaedics device according to claim 1 in which at least one of said first and second attachment means comprises:
a base plate for fixation to a vertebra; and
a connecting means for attaching said unidirectional force generating means to said base plate,
wherein said base plate is formed such that said connecting means can be connected at various locations on said base plate.

6. An orthopaedics device according to claim 1 in which at least one of said first and second attachment means comprises a plate for fixation to a vertebra, which plate comprises a plurality of connecting means for attaching said unidirectional force generating means to said plate at a variety of locations on said plate.

7. An orthopaedics device according to either of claim 5 or claim 6 in dependence on claim 4 in which said mobile joint is provided by an interface between said unidirectional force generating means and said connecting means.

8. An orthopaedics device according to claim 1 in which said unidirectional force generating means is at least partially formed out of a biocompatible, superelastic shape memory alloy, such as a Ni—Ti shape memory alloy.

9. An orthopaedics device according to claim 1 in which said unidirectional force generating means is a spring.

10. An orthopaedics device according to claim 9 in which said spring is a conventional, coiled spring which generates said unidirectional force by the application of torsional deformation perpendicularly on a coil or a plurality of coils of the coiled spring.

11. An orthopaedics device according to claim 9 in which said spring is a bending spring which generates said unidirectional force by the application of bending moments on curves of the bending spring.

12. An orthopaedics device according to claim 11 in which said bending spring comprises a length of elastic or super-elastic material shaped into at least one C- or S-shaped curve at at least one point along its length.

13. An orthopaedics device according to claim 9 in which said unidirectional force is generated by setting said spring in tension or compression between said first and second attachment points during the course of implantation.

14. An orthopaedics device according to claim 13 in which a plurality of said springs are provided such that a setting of the magnitude of said unidirectional force is achieved by appropriate pre- or intra-operative selection of a spring from said plurality of springs.

15. An orthopaedics device according to claim 13 in which at least one of said first and second attachment means comprises releasable clamping means for releasably clamping said spring to said first and/or said second attachment means, wherein said tension or compression is achieved through i) releasing said releasable clamping means, ii) mechanically applying said tension or compression and iii) clamping said releasable clamping means.

16. An orthopaedics device according to claim 13 in which at least one of said first and second attachment means comprises unidirectional gripping means which allow motion of said spring in relation to said attachment means in one axial direction of said spring, but prevent such motion in the opposite axial direction of said spring, wherein said tension or compression is achieved through pushing or pulling said spring through said unidirectional gripping means.

17. An orthopaedics device according to claim 13 in which said spring is at least partially formed out of a biocompatible, superelastic shape memory alloy, such as a Ni-Ti shape memory alloy wherein said setting of said spring in tension or compression is achieved through a martensitic or austenitic transformation in the shape memory alloy section of the spring due to a difference between the pre-operative temperature of said spring and the intra- and/or post-operative temperature of said spring.

18. An orthopaedics device according to claim 1 in which said unidirectional force generating means is arranged such that said unidirectional force drops to substantially zero in the proximity of a position at which said first and second vertebrae attain said predetermined desired spatial relationship.

19. An orthopaedics device according to claim 1 in which said unidirectional force generating means is shaped either at manufacture or intraoperatively to substantially conform to the shape of the portions of the surfaces of said first and second vertebrae over which said unidirectional force generating means passes.

20. An implantable, temporospatially dynamic, rachiorthotic orthopaedics system comprising a plurality of orthopaedics devices according to any of the preceding claims, wherein the orientation of the unidirectional force generating means of one of said plurality of orthopaedics devices may be set independently of the setting of the orientation of the unidirectional force generating means of at least one other of said plurality of orthopaedics devices.

21. An implantable, hybrid static/dynamic rachiorthotic orthopedics system comprising an orthopaedics system according to claim 20 and a rod or rods which may be attached in the place of at least one of said unidirectional force generating means by using at least part of said attachment means, whereby a choice may be made pre and/or intraoperatively for each motion segment which is to be treated whether to apply static or dynamic methods.

22. An orthopaedic implantation kit comprising an orthopaedics device according to claim 1 or a system according to claim 20 and further comprising pre-operative planning means comprising of computer software which suggests appropriate orientation(s), points of attachment and/or force(s) for said unidirectional force generating means.

23. An orthopaedic implantation kit according to claim 22 in which said computer software utilises a finite element model of the spine in generating said suggestions.

24. An orthopaedic implantation kit according to claim 22 in which said computer software utilises data gathered from a digitised X-ray of vertebrae which are to be treated.
